(19) United States
(12) Patent Application Publication
(10) Pub. No.: US 2011/0054611 A1
(43) Pub. Date: Mar. 3, 2011

(54) Implant Structure

(75) Inventors: Ming-Chang Wu, Guishan Shiang (TW); Jia-Jyun Hong, Guishan Shiang (TW); Chi-Bin Wu, Guishan Shiang (TW)

(73) Assignee: AccuMIS Inc., Zhonghe City (TW)

(21) Appl. No.: 12/619,892

(22) Filed: Nov. 17, 2009

(30) Foreign Application Priority Data
Aug. 31, 2009 (TW) 098129177

(51) Int. Cl. A61F 2/28

(52) U.S. Cl. 623/16.11

(57) ABSTRACT

An implant structure for medical use is provided. The implant structure includes an implant body and a nailing portion. The implant body and the nailing portion are integrally formed as a unitary structure to thereby prevent separation between the implant body and the nailing portion which might otherwise occur after long use. The nailing portion has a bone-contacting surface with at least one barb unit formed thereon for enhancing the bonding of the implant structure and a bone, preventing the implant structure from loosening, and extending the service life of the implant structure.

### BACKGROUND OF THE INVENTION

1. Technical Field

The present invention relates to medical implant structures. More particularly, the present invention relates to an implant structure for medical repair and fixation of damaged bones.

2. Description of Related Art

Owing to advancements in medical technology, an ageing society is here with us today. With an ever-increasing ageing population, geriatric care and medical treatment for chronic diseases are becoming more important.

In such an ageing society, degenerative arthritis, which is common among people over 50, is drawing attention. The elderly with severe degenerative arthritis usually have aching knees and thus are crippled to the detriment of their daily routine.

Degenerative arthritis develops because of wear and tear of the cartilage of a joint and ends up with the deformed joint and cracks on the surface of the bone involved. Hence, degenerative arthritis is treated with medication or surgery, depending on the damage done to the bone. Where a joint affected by degenerative arthritis is worn out to the extent that medication or rehabilitative therapy is inadequate to alleviate symptoms of degenerative arthritis, a surgeon replaces the damaged joint with an implant to restore joint functions.

FIG. 1 is a perspective view of a conventional implant structure 10. FIG. 2 is an exploded perspective view of the conventional implant structure 10. Referring to FIG. 1 and FIG. 2, the conventional implant structure 10 comprises a fixing mechanism 11, a coupling mechanism 12, and an implant mechanism 13. The fixing mechanism 11 is configured to be implanted in a damaged bone. After the fixing mechanism 11 has been fixed in position to the bone, the implant mechanism 13 can be coupled to the fixing mechanism 11 by means of the coupling mechanism 12, such that the implant structure 10 is fixed in position to the damaged bone.

Human joints are evolved to be heavy-duty in terms of frequency of use and directions of applied forces. Hence, given time, the fixing mechanism 11 of the implant structure 10 implanted in the bone of a patient is likely to be loosened and displaced under the forces exerted upon the implant structure 10 (which acts as a substitute for the otherwise damaged joint of a patient), and the coupling mechanism 12 may deform so that the implant mechanism 13 becomes disconnected from the fixing mechanism 11. Thus, the service life of the implant structure 10 is shortened significantly.

Furthermore, in situations where the implant mechanism 13 of the implant structure 10 is relatively large, a plurality of said fixing mechanisms 11 are essential to enhancement of the bonding of the implant structure 10 and the damaged bone. In such situations, accurate alignment of the implant mechanism 13 and the fixing mechanism 11 is of vital importance, and as a consequence, it is rather difficult to implant the implant structure 10 in the damaged bone.

### SUMMARY OF THE INVENTION

The present invention provides an implant structure having a nailing portion formed with a barb unit for enhancing the bonding between the implant structure and a bone to thereby prevent the implant structure from displacement.

The present invention provides an implant structure having an implant body and a nailing portion, wherein the implant body and the nailing portion are integrally formed as a unitary structure to thereby prevent separation of the implant body and the nailing portion.

The present invention provides an implant structure having an implant body and a nailing portion, wherein the implant body and the nailing portion are integrally formed as a unitary structure to thereby streamline implantation of the implant structure in a bone; hence, implantation of the implant structure is simplified.

To achieve the above and other objectives, the present invention provides an implant structure, comprising: an implant body having a first surface, a second surface, and a third surface, with the first and second surfaces adjoining via a rim shared therebetween and the second and third surfaces adjoining via a rim shared therebetween; and at least one nailing portion having a bone-contacting surface with at least one barb unit formed thereon, wherein the implant body and the said nailing portion are integrally formed as a unitary structure.

Implementation of the present invention involves at the least the inventive steps as follows:

1. With the implant body and the nailing portion being integrally formed as a unitary structure, separation of the implant body and the nailing portion is prevented.

2. The nailing portion is formed with the barb unit for enhancing the bonding of the implant structure and a bone to thereby prevent the implant structure from loosening or displacement.

3. With the implant body and the nailing portion being integrally formed as a unitary structure in advance, implantation is simplified.

### BRIEF DESCRIPTION OF THE DRAWINGS

The features and advantages of present invention are described in detail hereunder to enable persons skilled in the art to understand and implement the disclosure of the present invention and readily apprehend objectives and advantages of the present invention with references made to the disclosure contained in the specification, the claims, and accompanying drawings, wherein:

FIG. 1 is a perspective view of a conventional implant structure;

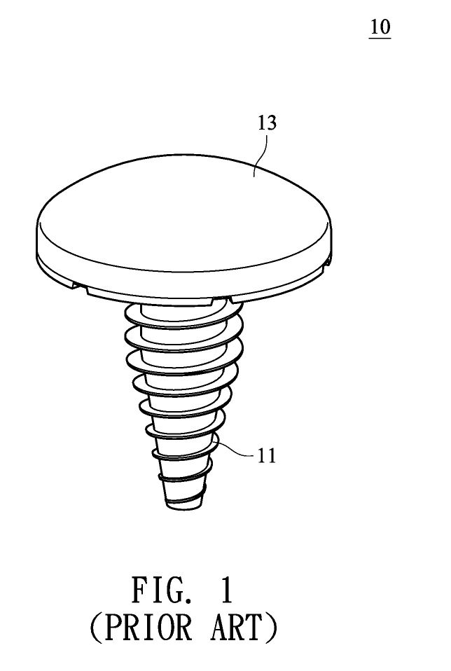

FIG. 2 is an exploded perspective view of the conventional implant structure;

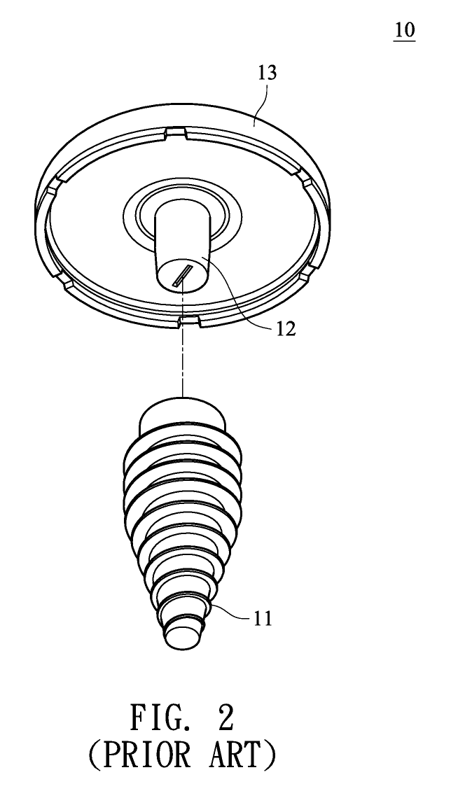

FIG. 3 is a perspective view of a first embodiment of an implant structure according to the present invention;

FIG. 4 is a perspective view of a second embodiment of the implant structure according to the present invention; and

FIG. 5 is a perspective view of a third embodiment of the implant structure according to the present invention.

### DETAILED DESCRIPTION OF THE PREFERRED EMBODIMENTS

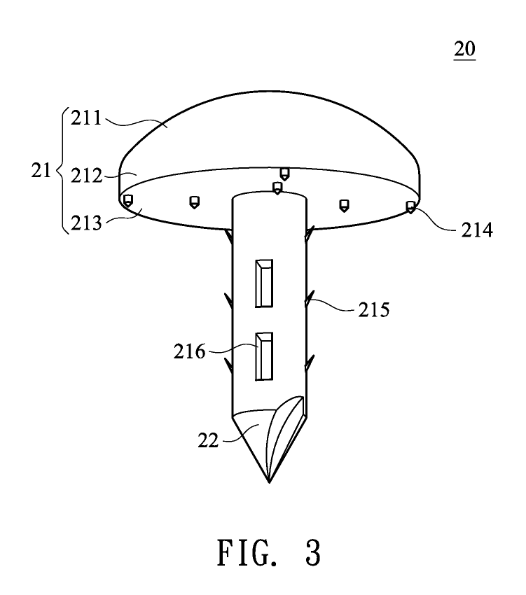

Referring to FIG. 3, there is shown a perspective view of a first embodiment of an implant structure 20 according to the present invention. As shown in the drawing, the implant structure 20 includes an implant body 21 and at least one nailing portion 22.

Referring to FIG. 3, the implant body 21 has a first surface 211, a second surface 212, and a third surface 213. After the implant body 21 has been implanted in a bone, the first surface 211 defines the bone-bone contact surface of a joint implanted with the implant structure 20. Hence, the profile of the first surface 211 is designed as needed, so as to maintain the flatness of the surface of the bone repaired. The second surface 212 and the first surface 211 adjoin via a rim shared therebetween, in order that the implant body 21 to have a certain thickness. The third surface 213 and the second surface 212 adjoin via a rim shared therebetween, so that the implant body 21 has a bottom side. In the first embodiment, the second surface 212 and the third surface 213 are perpendicular to each other, and the implant body 21 is made of alloy or metal.

Referring to FIG. 3, the third surface 213 of the implant body 21 is further provided with a plurality of fixing units 214 thereon. The fixing units 214 are needle-shaped and thereby assist in fixing the third surface 213 to the surface of the bone and enhancing the bonding between the implant structure 20 and the bone. Likewise, the fixing units 214 are made of alloy or metal.

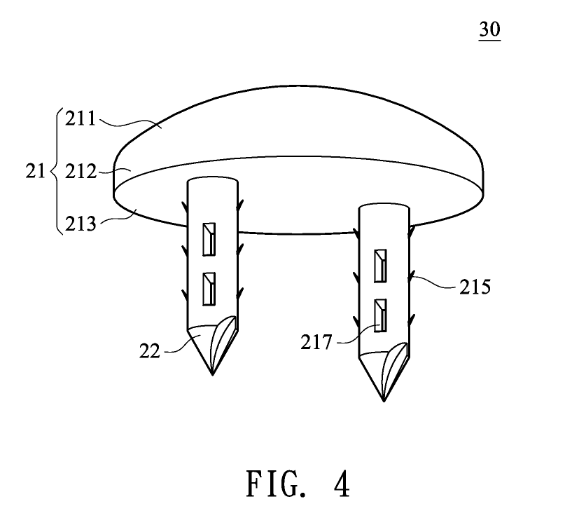

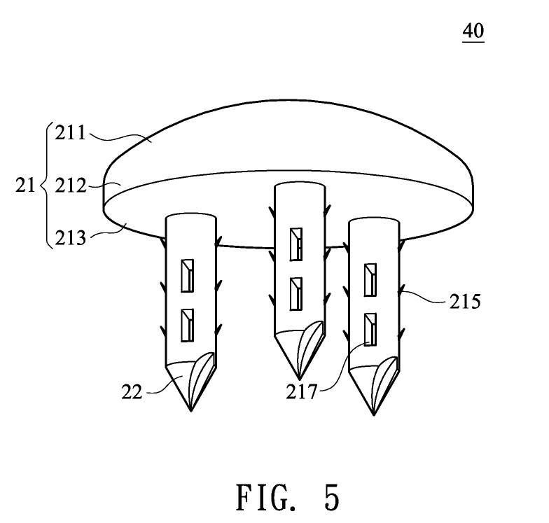

Referring to FIG. 3, the nailing portion 22 and the implant body 21 are integrally formed as a unitary structure. The nailing portion 22 is formed on and extends from the third surface 213. The nailing portion 22 has a bone-contacting surface with at least one barb unit 215 formed thereon. Referring to FIG. 4 and FIG. 5, there are shown perspective views of second and third embodiments of the implant structure according to the present invention, respectively. As shown in the drawings, implant structures 30 and 40 comprise two said nailing portions 22 and three said nailing portions 22, respectively. Each of the nailing portions 22 of the implant structures 30 and 40 has a bone-contacting surface with the at least one barb unit 215 formed thereon. When two or more said barb units 215 are provided, the barb units 215 are preferably positioned on two opposing sides of the nailing portion 22 for improved fixing.

To be implanted in a bone, the implant structure 20 that comes in a single piece is not screwed into the bone as taught by the prior art but is directly nailed into the bone. Hence, the at least one barb unit 215 on the bone-contacting surface of the nailing portion 22 engage with the bone so as to counterbalance an upward pull exerted upon the implant structure 20. Also, with the implant structure 20 being a unitary structure, separation between the implant body 21 and the nailing portion 22 is efficiently prevented.

Referring to FIG. 4 and FIG. 5, with the two said nailing portions 22 and three said nailing portions 22, respectively situated on the implant structures 30 and 40, the latter serves as artificial joints. Thus, the implant structures 30 and 40 can better oppose a rotational force exerted thereon and thus are unlikely to loosen. Since the implant body 21 and the nailing portions 22 of each of the implant structures 30 and 40 are integrally formed as a unitary structure, artificial joint implantation surgery is easier than ever before, and the resultant bonding between the implant structures 30 and 40 with the bone is stronger than ever before.

Referring to FIG. 3 through FIG. 5, each of the nailing portions 22 of the implant structures 20, 30, and 40 is further provided with at least one recess 216 or at least one cavity 217. The said recess 216 or the said cavity 217 serves to receive a metal frame (not shown). The metal frame is coated with hydroxylapatite for providing a favorable growth environment for the bone with a view to promoting osteogenesis and enhancing the bonding between the implant structures 20, 30, 40 and the bone.

The foregoing descriptions of the embodiments are provided to illustrate the features of the present invention and enable those in the art to gain insight into the present invention and implement the present invention, but are not intended to restrict the scope of the present invention. It should be understood by those in the art that equivalent modifications and variations in the aforesaid embodiments of the present invention can be made without departing from the spirit and principle in the disclosure of the present invention and still fall within the scope of the invention as set forth in the appended claims.

What is claimed is:

1. An implant structure, comprising:
an implant body having a first surface, a second surface, and a third surface, with the first and second surfaces adjoining via a rim shared therebetween, and the second and third surfaces adjoining via a rim shared therebetween; and
at least one nailing portion having a bone-contacting surface with at least one barb unit formed thereon;
wherein the implant body and the at least one nailing portion are integrally formed as a unitary structure.

2. The implant structure of claim 1, wherein the implant body is made of alloy or metal.

3. The implant structure of claim 1, wherein the said nailing portion is made of alloy or metal.

4. The implant structure of claim 1, wherein the second surface is perpendicular to the third surface.

5. The implant structure of claim 1, wherein the third surface is further provided with a plurality of fixing units thereon.

6. The implant structure of claim 5, wherein the fixing units are needle-shaped.

7. The implant structure of claim 5, wherein the fixing units are made of alloy or metal.

8. The implant structure of claim 1, wherein the said nailing portion is further provided with at least one recess.

9. The implant structure of claim 1, wherein the said nailing portion is further provided with at least one cavity.
